(19) United States
(12) Patent Application Publication
(10) Pub. No.: US 2011/0071400 A1
(43) Pub. Date: Mar. 24, 2011

(54) SYSTEMS AND METHODS FOR MAKING AND USING INTRAVASCULAR ULTRASOUND IMAGING SYSTEMS WITH SEALED IMAGING CORES

(75) Inventors: Roger N. Hastings, Maple Grove, MN (US); Tat-Jin Teo, Sunnyvale, CA (US); Kevin D. Edmunds, Ham Lake, MN (US); Michael J. Pikus, Golden Valley, MN (US); Leonard B. Richardson, Brooklyn Park, MN (US)

(73) Assignee: Boston Scientific SciMed, Inc., Maple Grove, MA (US)

(21) Appl. No.: 12/565,632

(22) Filed: Sep. 23, 2009

(51) Int. Cl. A61B 8/14

(52) U.S. Cl. 600/467

(57) ABSTRACT

A catheter assembly for an intravascular ultrasound system includes a catheter insertable into patient vasculature via a guidewire. A watertight imaging core is disposed in the distal end of the catheter. The imaging core includes a motor, at least one fixed transducer, and a signal redirection unit. The motor includes a magnet configured and arranged to rotate upon generation of a magnetic field by magnetic field windings. The signal redirection unit is coupled to the magnet such that rotation of the magnet causes a corresponding rotation of at least a portion of the signal redirection unit. The signal redirection unit includes a tilted mirror that redirects acoustic signals transmitted from the fixed transducer to patient tissue. At least one transducer conductor and at least one stator conductor are electrically coupled to the imaging core and in electrical communication with the proximal end of the catheter.

### TECHNICAL FIELD

The present invention is directed to the area of intravascular ultrasound imaging systems and methods of making and using the systems. The present invention is also directed to ultrasound imaging systems having motors disposed within sealed imaging cores, as well as methods for making and using the motors, sealed imaging cores, and intravascular ultrasound systems.

### BACKGROUND

Intravascular ultrasound (“IVUS”) imaging systems have proven diagnostic capabilities for a variety of diseases and disorders. For example, IVUS imaging systems have been used as an imaging modality for diagnosing blocked blood vessels and providing information to aid medical practitioners in selecting and placing stents and other devices to restore or increase blood flow. IVUS imaging systems have been used to diagnose atheromatous plaque build-up at particular locations within blood vessels. IVUS imaging systems can be used to determine the existence of an intravascular obstruction or stenosis, as well as the nature and degree of the obstruction or stenosis. IVUS imaging systems can be used to visualize segments of a vascular system that may be difficult to visualize using other intravascular imaging techniques, such as angiography, due to, for example, movement (e.g., a beating heart) or obstruction by one or more structures (e.g., one or more blood vessels not desired to be imaged). IVUS imaging systems can be used to monitor or assess ongoing intravascular treatments, such as angiography and stent placement in real (or almost real) time. Moreover, IVUS imaging systems can be used to monitor one or more heart chambers.

IVUS imaging systems have been developed to provide a diagnostic tool for visualizing a variety is diseases or disorders. An IVUS imaging system can include a control module (with a pulse generator, an image processor, and a monitor), a catheter, and one or more transducers disposed in the catheter. The transducer-containing catheter can be positioned in a lumen or cavity within, or in proximity to, a region to be imaged, such as a blood vessel wall or patient tissue in proximity to a blood vessel wall. The pulse generator in the control module generates electrical pulses that are delivered to the one or more transducers and transformed to acoustic pulses that are transmitted through patient tissue. Reflected pulses of the transmitted acoustic pulses are absorbed by the one or more transducers and transformed to electric pulses. The transformed electric pulses are delivered to the image processor and converted to an image displayable on the monitor.

### BRIEF SUMMARY

In one embodiment, a catheter assembly for an intravascular ultrasound system includes a catheter having a length, a distal end, and a proximal end. The distal end is configured and arranged for insertion into patient vasculature. A sealed imaging core is disposed in the distal end of the catheter. The sealed imaging core is configured and arranged to provide a watertight environment within the sealed imaging core. The sealed imaging core has a proximal end, a distal end, and a length that is substantially less than the length of the catheter. The sealed imaging core includes a motor, at least one fixed transducer, a tilted mirror, and at least one sonolucent fluid. The motor includes a magnet and at least two magnetic field windings. The magnet is configured and arranged to rotate upon generation of a magnetic field by the at least two magnetic field windings. The at least one fixed transducer is configured and arranged for transforming applied electrical signals to acoustic signals, transmitting the acoustic signals, receiving corresponding echo signals, and transforming the received echo signals to electrical signals. The tilted mirror is coupled to the magnet such that rotation of the magnet causes a corresponding rotation of the tilted mirror. The tilted mirror is configured and arranged to redirect acoustic signals transmitted from the at least one fixed transducers to patient tissue. The at least one sonolucent fluid is disposed in the sealed imaging core and fills open space within the sealed imaging core. At least one transducer conductor is electrically coupled to the at least one fixed transducer within the sealed imaging core. The at least one transducer conductor extends from the at least one fixed transducer to a location that is external to the sealed imaging core. The at least one stator conductor is electrically coupled to the magnetic field windings within the sealed imaging core. The at least one stator conductor extends from the magnetic field windings to a location that is external to the sealed imaging core.

In another embodiment, a catheter assembly for an intravascular ultrasound system includes a catheter having a length, a distal end, and a proximal end. The distal end is configured and arranged for insertion into patient vasculature via a guidewire. An imaging core is disposed in the distal end of the catheter. The imaging core is configured and arranged to provide a watertight environment within the imaging core. The imaging core has a proximal end, a distal end, and a length that is substantially less than the length of the catheter. The imaging core includes a motor, at least one fixed transducer, and a signal redirection unit. The motor includes a magnet and at least two magnetic field windings. The magnet is configured and arranged to rotate upon generation of a magnetic field by the at least two magnetic field windings. The magnet has an inner surface at an inner diameter and an outer surface at an outer diameter. The inner diameter is defined by an aperture along a longitudinal axis of the at least one transducer. The magnet aperture is configured and arranged to allow passage of the guidewire. The at least two magnetic field windings are disposed around at least a portion of both the inner surface and the outer surface of the magnet. The at least one fixed transducer has an aperture defined along a longitudinal axis of the at least one transducer. The at least one fixed transducer aperture is configured and arranged to allow passage of the guidewire. The at least one fixed transducer is configured and arranged for transforming applied electrical signals to acoustic signals, transmitting the acoustic signals, receiving corresponding echo signals, and transforming the received echo signals to electrical signals. The signal redirection unit is coupled to the magnet such that rotation of the magnet causes a corresponding rotation of at least a portion of the signal redirection unit. The signal redirection unit includes a tilted mirror configured and arranged to redirect acoustic signals transmitted from the at least one fixed transducers to patient tissue. At least one transducer conductor is electrically coupled to the at least one transducer and in electrical communication with the proximal end of the catheter. At least one stator conductor is electrically coupled to the magnetic field windings and in electrical communication with the proximal end of the catheter.

In yet another embodiment, a method for imaging a patient using an intravascular ultrasound imaging system includes inserting a catheter into patient vasculature. The catheter includes a sealed, watertight imaging core coupled to the guidewire. The imaging core is electrically coupled to a control module by at least one transducer conductor. The imaging core has at least one fixed transducer and a magnet that rotates by application of a magnetic field generated from at least two magnetic field windings. The magnet has an inner surface at an inner diameter and an outer surface at an outer diameter. The at least two magnetic field windings are disposed around at least a portion of both the inner surface and the outer surface of the magnet. The transducer emits acoustic signals directed at a tilted mirror configured and arranged to rotate with the magnet and redirect the acoustic signals to patient tissue. At least one electrical signal is transmitted from the control module to the at least one transducer. A magnetic field is generated to cause the magnet to rotate. At least one acoustic signal is transmitted from the at least one transducer to the tilted mirror. At least one echo signal received from a tissue-boundary between adjacent imaged patient tissue is redirected to the at least one transducer by the tilted mirror. At least one transformed echo signal is transmitted from the at least one transducer to the control module for processing.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an intravascular ultrasound imaging system, according to the invention;

FIG. 2 is a schematic side view of one embodiment of a catheter of an intravascular ultrasound imaging system, according to the invention;

FIG. 3 is a schematic perspective view of one embodiment of a distal end of the catheter shown in FIG. 2 with an imaging core disposed in a lumen defined in the catheter, according to the invention;

FIG. 4 is a schematic longitudinal cross-sectional view of one embodiment of a catheter assembly including a sealed imaging core disposed in a catheter and a guidewire extending through the sealed imaging core, according to the invention;

FIG. 5A is a schematic transverse cross-sectional view of one embodiment of the guidewire of FIG. 4 extending through a transducer disposed in the imaging core of FIG. 4 such that a blind spot is formed by the guidewire during imaging, according to the invention;

FIG. 5B is a schematic transverse cross-sectional view of one embodiment of the guidewire, transducer, and blind spot of FIG. 5A, the blind spot rotated ninety degrees from the location shown in FIG. 5A, according to the invention;

FIG. 6A is a schematic perspective view of one embodiment of a magnetic field winding suitable for use with the imaging core of FIG. 4, according to the invention;

FIG. 6B is a schematic end view of one embodiment of the magnet of FIG. 4 disposed in the magnetic field winding of FIG. 4, according to the invention;

FIG. 7 is a schematic longitudinal cross-sectional view of one embodiment of a sealed imaging core disposed in a lumen of a catheter, the imaging core including a fixed transducer and a rotatable mirror, according to the invention;

FIG. 8 is a schematic longitudinal cross-sectional view of another embodiment of a sealed imaging core disposed in a lumen of a catheter, the imaging core including a fixed transducer and a rotatable mirror, according to the invention; and

FIG. 9 is a schematic longitudinal cross-sectional view of one embodiment of a sealed imaging core disposed in a lumen of a catheter, the imaging core including a rotatable transducer, according to the invention.

### DETAILED DESCRIPTION

The present invention is directed to the area of intravascular ultrasound imaging systems and methods of making and using the systems. The present invention is also directed to ultrasound imaging systems having motors disposed within sealed imaging cores, as well as methods for making and using the motors, sealed imaging cores, and intravascular ultrasound systems.

Suitable intravascular ultrasound (“IVUS”) imaging systems include, but are not limited to, one or more transducers disposed on a distal end of a catheter configured and arranged for percutaneous insertion into a patient. Examples of IVUS imaging systems with catheters are found in, for example, U.S. Pat. Nos. 7,306,561; and 6,945,938; as well as U.S. Patent Application Publication Nos. 20060253028; 20070016054; 20070038111; 20060173350; and 20060100522, all of which are incorporated by reference.

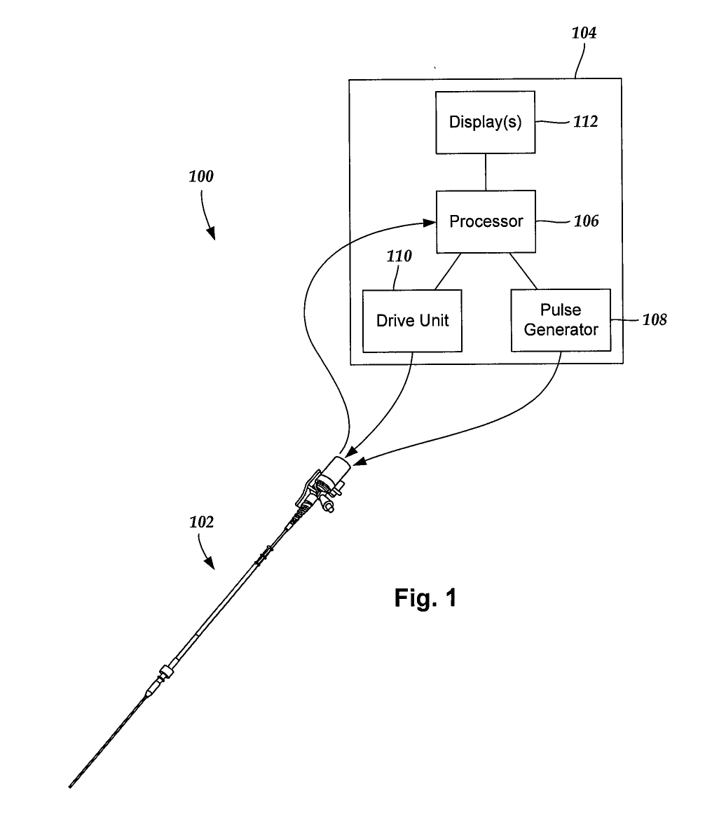

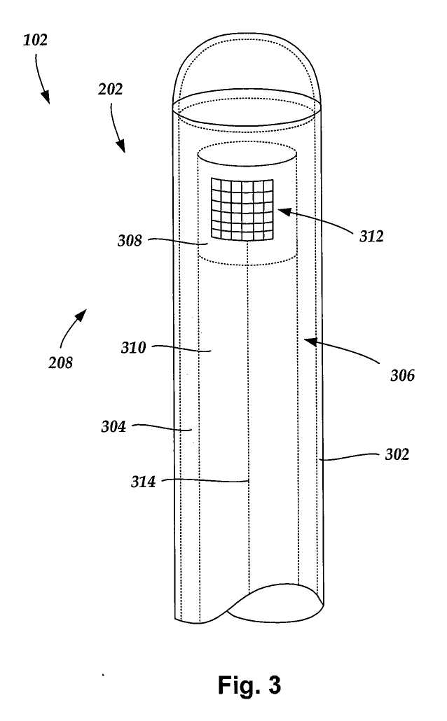

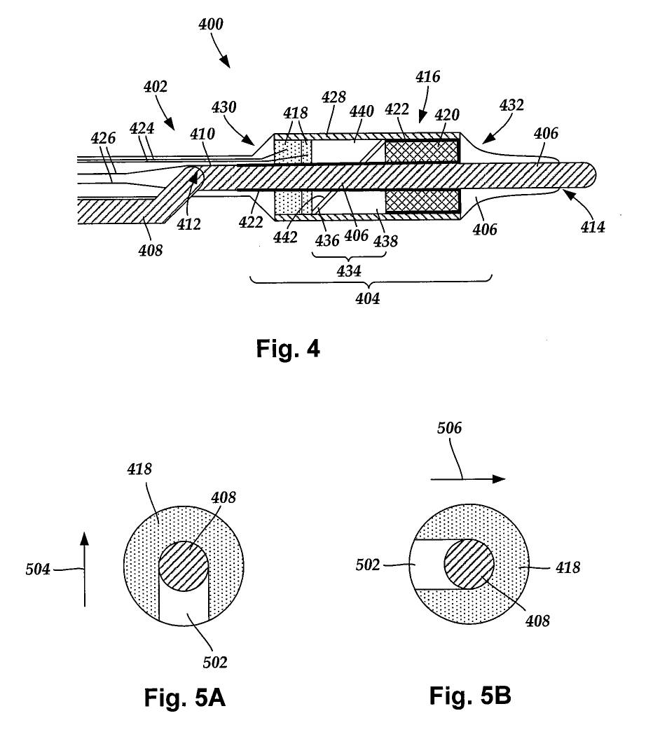

FIG. 1 illustrates schematically one embodiment of an IVUS imaging system 100. The IVUS imaging system 100 includes a catheter 102 that is coupleable to a control module 104. The control module 104 may include, for example, a processor 106, a pulse generator 108, a drive unit 110, and one or more displays 112. In at least some embodiments, the pulse generator 108 forms electric pulses that may be input to one or more transducers (312 in FIG. 3) disposed in the catheter 102. In at least some embodiments, signals from the drive unit 110 may be used to control a motor (see e.g., 416 in FIG. 4) driving an imaging core (306 in FIG. 3) disposed in the catheter 102. In at least some embodiments, electric pulses transmitted from the one or more transducers (312 in FIG. 3) may be input to the processor 106 for processing. In at least some embodiments, the processed electric pulses from the one or more transducers (312 in FIG. 3) may be displayed as one or more images on the one or more displays 112. In at least some embodiments, the processor 106 may also be used to control the functioning of one or more of the other components of the control module 104. For example, the processor 106 may be used to control at least one of the frequency or duration of the electrical pulses transmitted from the pulse generator 108, the rotation rate of the imaging core (306 in FIG. 3) by the motor, the velocity or length of the pullback of the imaging core (306 in FIG. 3) by the motor, or one or more properties of one or more images formed on the one or more displays 112.

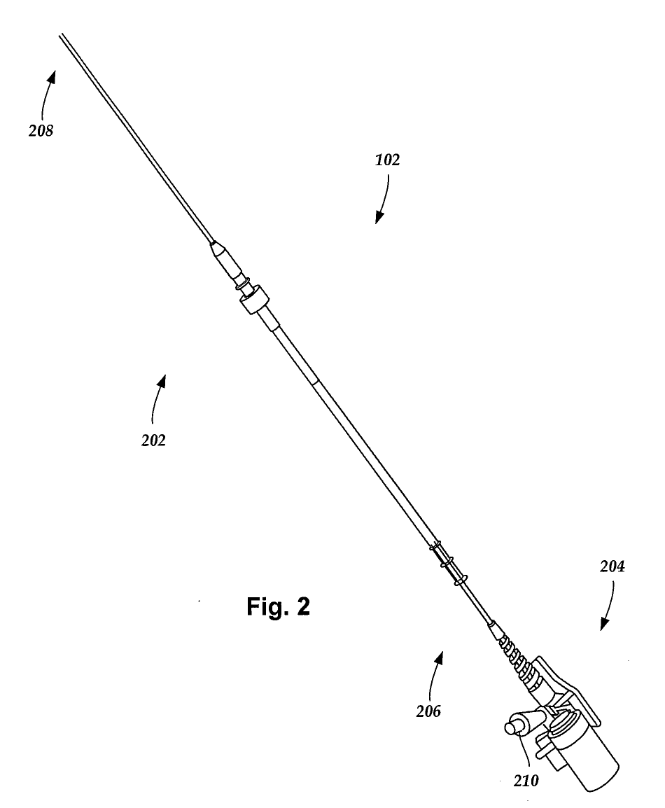

FIG. 2 is a schematic side view of one embodiment of the catheter 102 of the IVUS imaging system (100 in FIG. 1). The catheter 102 includes an elongated member 202 and a hub 204. The elongated member 202 includes a proximal end 206 and a distal end 208. In FIG. 2, the proximal end 206 of the elongated member 202 is coupled to the catheter hub 204 and the distal end 208 of the elongated member is configured and arranged for percutaneous insertion into a patient. In at least some embodiments, the catheter 102 defines at least one flush port, such as flush port 210. In at least some embodiments, the flush port 210 is defined in the hub 204. In at least some embodiments, the hub 204 is configured and arranged to couple to the control module (104 in FIG. 1). In some embodiments, the elongated member 202 and the hub 204 are formed as a unitary body. In other embodiments, the elongated member 202 and the catheter hub 204 are formed separately and subsequently assembled together.

FIG. 3 is a schematic perspective view of one embodiment of the distal end 208 of the elongated member 202 of the catheter 102. The elongated member 202 includes a sheath 302 and a lumen 304. An imaging core 306 is disposed in the lumen 304. The imaging core 306 includes an imaging device 308 coupled to a distal end of a rotatable driveshaft 310.

The sheath 302 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

One or more transducers 312 may be mounted to the imaging device 308 and employed to transmit and receive acoustic pulses. In a preferred embodiment (as shown in FIG. 3), an array of transducers 312 are mounted to the imaging device 308. In other embodiments, a single transducer may be employed. In yet other embodiments, multiple transducers in an irregular-array may be employed. Any number of transducers 312 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used.

The one or more transducers 312 may be formed from one or more known materials capable of transforming applied electrical pulses to pressure distortions on the surface of the one or more transducers 312, and vice versa. Examples of suitable materials include piezoelectric ceramic materials, piezocomposite materials, piezoelectric plastics, barium titanates, lead zirconate titanates, lead metaniobates, polyvinylidenefluorides, and the like.

The pressure distortions on the surface of the one or more transducers 312 form acoustic pulses of a frequency based on the resonant frequencies of the one or more transducers 312. The resonant frequencies of the one or more transducers 312 may be affected by the size, shape, and material used to form the one or more transducers 312. The one or more transducers 312 may be formed in any shape suitable for positioning within the catheter 102 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 312 may include a layer of piezoelectric material sandwiched between a conductive acoustic lens and a conductive backing material formed from an acoustically absorbent material (e.g., an epoxy substrate with tungsten particles). During operation, the piezoelectric layer may be electrically excited by both the backing material and the acoustic lens to cause the emission of acoustic pulses.

In at least some embodiments, the one or more transducers 312 can be used to form a radial cross-sectional image of a surrounding space. Thus, for example, when the one or more transducers 312 are disposed in the catheter 102 and inserted into a blood vessel of a patient, the one more transducers 312 may be used to form an image of the walls of the blood vessel and tissue surrounding the blood vessel.

In at least some embodiments, the imaging core 306 may be rotated about a longitudinal axis of the catheter 102. As the imaging core 306 rotates, the one or more transducers 312 emit acoustic pulses in different radial directions. When an emitted acoustic pulse with sufficient energy encounters one or more medium boundaries, such as one or more tissue boundaries, a portion of the emitted acoustic pulse is reflected back to the emitting transducer as an echo pulse. Each echo pulse that reaches a transducer with sufficient energy to be detected is transformed to an electrical signal in the receiving transducer. The one or more transformed electrical signals are transmitted to the control module (104 in FIG. 1) where the processor 106 processes the electrical-signal characteristics to form a displayable image of the imaged region based, at least in part, on a collection of information from each of the acoustic pulses transmitted and the echo pulses received. In at least some embodiments, the rotation of the imaging core 306 is driven by the motor (see e.g., 416 in FIG. 4).

As the one or more transducers 312 rotate about the longitudinal axis of the catheter 102 emitting acoustic pulses, a plurality of images are formed that collectively form a radial cross-sectional image of a portion of the region surrounding the one or more transducers 312, such as the walls of a blood vessel of interest and the tissue surrounding the blood vessel. In at least some embodiments, the radial cross-sectional image can be displayed on one or more displays 112.

In at least some embodiments, the imaging core 306 may also move longitudinally along the blood vessel within which the catheter 102 is inserted so that a plurality of cross-sectional images may be formed along a longitudinal length of the blood vessel. In at least some embodiments, during an imaging procedure the one or more transducers 312 may be retracted (i.e., pulled back) along the longitudinal length of the catheter 102. In at least some embodiments, the catheter 102 includes at least one telescoping section that can be retracted during pullback of the one or more transducers 312. In at least some embodiments, the motor (see e.g., 416 in FIG. 4) drives the pullback of the imaging core 306 within the catheter 102. In at least some embodiments, the motor pullback distance of the imaging core is at least 5 cm. In at least some embodiments, the motor pullback distance of the imaging core is at least 10 cm. In at least some embodiments, the motor pullback distance of the imaging core is at least 15 cm. In at least some embodiments, the motor pullback distance of the imaging core is at least 20 cm. In at least some embodiments, the motor pullback distance of the imaging core is at least 25 cm.

The quality of an image produced at different depths from the one or more transducers 312 may be affected by one or more factors including, for example, bandwidth, transducer focus, beam pattern, as well as the frequency of the acoustic pulse. The frequency of the acoustic pulse output from the one or more transducers 312 may also affect the penetration depth of the acoustic pulse output from the one or more transducers 312. In general, as the frequency of an acoustic pulse is lowered, the depth of the penetration of the acoustic pulse within patient tissue increases. In at least some embodiments, the IVUS imaging system 100 operates within a frequency range of 5 MHz to 60 MHz.

In at least some embodiments, one or more transducer conductors 314 electrically couple the transducers 312 to the control module 104 (See FIG. 1). In at least some embodiments, the one or more transducer conductors 314 extend along a longitudinal length of the rotatable driveshaft 310.

In at least some embodiments, the catheter 102, with one or more transducers 312 mounted to the distal end 208 of the imaging core 308, may be inserted percutaneously into a patient via an accessible blood vessel, such as the femoral artery, at a site remote from the target imaging location. The catheter 102 may then be advanced through the blood vessels of the patient to the target imaging location, such as a portion of a selected blood vessel.

A catheter assembly includes a motor that is at least partially disposed in the imaging core. The motor includes a rotor and a stator. The rotor is a rotatable magnet and the stator includes a plurality of magnetic field windings configured and arranged to rotate the magnet by a generated magnetic field. Examples of IVUS imaging systems with motors that use rotatable magnets driven by magnetic field windings include, for example, U.S. patent application Ser. Nos. 12/415,724; 12/415,768; and 12/415,791, all of which are incorporated by reference.

In at least some embodiments, the magnetic field windings (“windings”) are also disposed in the imaging core. In alternate embodiments, the windings are disposed external to the catheter. In at least some embodiments, the windings are disposed external to a patient during an imaging procedure. In at least some embodiments, the imaging core is configured and arranged for coupling to a guidewire. In at least some embodiments, the imaging core is configured and arranged for insertion into the lumen of the catheter.

In at least some embodiments, the imaging core is configured and arranged such that rotation of the magnet causes a corresponding rotation of the one or more transducers configured and arranged to transmit energy to patient tissue and receive corresponding echo signals. In alternate embodiments, the one or more transducers do not rotate. Instead, the imaging core is configured and arranged such that rotation of the magnet causes a corresponding rotation of a tilted mirror configured and arranged to redirect energy between the one or more fixed transducers and patient tissue. Exemplary embodiments of imaging cores with a rotating mirror and fixed transducer are described below, with reference to FIGS. 4, 7 and 8. An exemplary embodiment of an imaging core with a rotating transducer is described above, with reference to FIG. 3. Additionally, other exemplary embodiments of imaging cores with rotating transducers are described below, with reference to FIGS. 9 and 10. In at least some embodiments, the motor is configured and arranged for rotating both the one or more transducers and the mirror.

Air in the imaging core may disrupt signal propagation between the one or more transducers and patient tissue. Thus, it is typically desirable to remove air from the imaging core prior to an imaging procedure. In some cases, open space within the imaging core is filled with a sonolucent fluid, such as a saline fluid, with impedance that matches (or nearly matches) patient tissue or fluids at or around a target imaging location. Air bubbles, however, may develop over time. Accordingly, some conventional IVUS systems may require one or more saline flushes be performed before, or even during, an imaging procedure to maintain an environment within a catheter that is conducive to signal propagation between the one or more transducers and patient tissue.

In order to reduce or prevent the formation of air bubbles in the imaging core, the catheter assembly includes a sealed and preferably watertight imaging core (“imaging core”). In at least some embodiments, the catheter assemblies utilize imaging cores that do not need to be flushed during an imaging procedure. In at least some embodiments, the imaging cores, once filled with a fluid and sealed, do not need to be flushed prior to an imaging procedure. The magnet is disposed in the imaging core. In at least some embodiments, the windings are also disposed in the imaging core. In at least some embodiments, when a mirror is used to redirect acoustic or echo signals, the mirror is disposed in the imaging core. An exemplary embodiment of an imaging core configured and arranged for being used in conjunction with a guidewire is described below, with reference to FIG. 4. Alternate embodiments of imaging cores for use without guidewires are described below, with reference to FIGS. 3 and 7-10.

FIG. 4 is a schematic longitudinal cross-sectional view of one embodiment of a catheter assembly 400 that includes a catheter 402 and a guidewire 408. An imaging core 404 is disposed within a body 406 of the catheter 402. In at least some embodiments, the imaging core 404 is disposed in proximity to a distal end of the catheter 402. In at least some embodiments, the imaging core 404 has a length that is substantially less than a length of the catheter 402.

The imaging core 404 is configured and arranged to be guided to a target imaging location by the guidewire 408. The catheter 402 can be configured and arranged to receive the guidewire 408, for example, via a guidewire lumen 410 defined along at least a portion of the catheter 402. In at least some embodiments, the guidewire lumen 410 extends from a proximal guidewire port 412 to a distal tip of the imaging core 404. In at least some embodiments, the guidewire lumen 410 defines a distal guidewire port 414 through which the guidewire 408 can extend. In at least some embodiments, the guidewire lumen 410 extends along at least a portion of the imaging core 404. In at least some embodiments, the guidewire lumen 410 extends along an entire length of the imaging core 404. In at least some embodiments, the guidewire lumen 410 extends along substantially entirely the length of the catheter 402. In at least some embodiments, the proximal guidewire port 412 is defined in a portion of the catheter 402 that is proximal to the imaging core 404. In at least some embodiments, the proximal guidewire port 412 is defined in a portion of the catheter 402 that is in proximity to a proximal end of the catheter 402. In at least some embodiments, the distal guidewire port 414 is defined in a portion of the imaging core 404. In at least some embodiments, the distal guidewire port 414 is defined in a portion of the catheter 402 that is distal to the imaging core 404.

The imaging core 404 includes a motor 416 and one or more transducers 418. The motor 416 includes a rotatable magnet 420 and windings 422. One or more transducer conductors 424 extend from the one or more transducers 418 along at least a portion of the catheter 402. In at least some embodiments, the one or more transducer conductors 424 electrically couple the one or more transducers 418 to the control module (104 in FIG. 1). In at least some embodiments, the one or more transducer conductors 424 extend along at least a portion of the catheter 402 as shielded electrical cables, such as a coaxial cable, or a twisted pair cable, or the like. In at least some embodiments, the one or more transducer conductors 424 may be attached to contacts on the distal end of the catheter 402 that, in turn, are connected to control module contacts.

One or more stator conductors 426 extend from the windings 422 along at least a portion of the catheter 402. In at least some embodiments, the one or more stator conductors 426 electrically couple the windings 422 to the control module (104 in FIG. 1). In at least some embodiments, the one or more stator conductors 426 may be attached to contacts on the distal end of the catheter 402 that, in turn, are connected to control module contacts. In at least some embodiments, at least a portion of the distal ends of the one or more transducer conductors 424 and the one or more stator conductors 426 are disposed in the imaging core 404 while at least a portion of the of the proximal ends of the one or more transducer conductors 424 and the one or more stator conductors 426 are disposed external to the imaging core 404. In at least some embodiments, the one or more transducer conductors 424 and the one or more stator conductors 426 extend through one or more sealed portions of the imaging core 404.

In at least some embodiments, a sonolucent sheath 428 radially surrounds the imaging core 404. In at least some embodiments, the sheath 428 is formed from one or more materials with an acoustic impedance that is within 20 percent of an acoustic impedance of patient tissue or fluid at the ultrasound imaging frequency, at or near a target imaging site within the patient. In at least some embodiments, the sheath 428 is formed from one or more materials with an acoustic impedance that is within 15 percent of an acoustic impedance of patient tissue or fluid at the ultrasound imaging frequency, at or near a target imaging site within the patient. In at least some embodiments, the sheath 428 is formed from one or more materials with an acoustic impedance that is within 10 percent of an acoustic impedance of patient tissue or fluid at the ultrasound imaging frequency, at or near a target imaging site within the patient. In at least some embodiments, the sheath 428 is formed from one or more materials with an acoustic impedance that is within 5 percent of an acoustic impedance of patient tissue or fluid at the ultrasound imaging frequency, at or near a target imaging site within the patient.

In at least some embodiments, the sheath 428 has a reduced level of attenuation of the ultrasound beam by virtue of a sufficiently thin wall. For example, a thin rigid tube made from a material such as polyimide, has reduced attenuation when the tube wall thickness is less than one wavelength of an ultrasound signal transmitting in patient fluid or tissue at the imaging frequency of the one or more transducers 418. In at least some embodiments, the sheath 428 has a wall thickness that is no more than, or less than one-half of the wavelength of an ultrasound signal transmitting in patient fluid or tissue at the imaging frequency of the one or more transducers 418. In at least some embodiments, the sheath 428 has a wall thickness that is no more than, or less than one-quarter of the wavelength of an ultrasound signal transmitting in patient fluid or tissue at the imaging frequency of the one or more transducers 418.

Without wishing to be held to any particular values, in one example ultrasound signals transmitting from the one or more transducers 418 have a frequency of 40 MHz, and the speed of sound in surrounding tissue is 1,500 m/sec, so the ultrasound wavelength is about 0.04 mm. In at least some embodiments, when the ultrasound wavelength is about 0.04 mm, the sheath 428 is constructed from polyimide having a wall thickness of no more than 0.04 mm. In at least some embodiments, when the ultrasound wavelength is about 0.04 mm, the sheath 428 is constructed from polyimide having a wall thickness of no more than 0.02 mm. In at least some embodiments, when the ultrasound wavelength is about 0.04 mm, the sheath 428 is constructed from polyimide having a wall thickness of no more than 0.01 mm.

In at least some embodiments, the sheath 428 is configured and arranged to provide cushion to the imaging core 404 when the imaging core 404 contacts patient tissue during an imaging procedure, thereby reducing the likelihood that the tissue contact will cause a non-uniform rotation of the magnet 420 (which may adversely affect images generated by the imaging procedure). In at least some embodiments, the imaging core 404 includes a tapered proximal end 430, a tapered distal end 432, or both. The one or more tapered ends 430 and 432 may facilitate axial movement of the imaging core 404 through patient vasculature.

In at least some embodiments, a sonolucent fluid is disposed in the imaging core 404 to displace open space within the imaging core 404, and also to lubricate interfaces between moving and non-moving components of the imaging core 404 during an imaging procedure. In at least some embodiments, the sonolucent fluid has an acoustic impedance that is within 20 percent of an acoustic impedance of patient tissue or fluid at or near a target imaging site within the patient. In at least some embodiments, the sonolucent fluid has an acoustic impedance that is within 15 percent of an acoustic impedance of patient tissue or fluid at or near a target imaging site within the patient. In at least some embodiments, the sonolucent fluid has an acoustic impedance that is within 10 percent of an acoustic impedance of patient tissue or fluid at or near a target imaging site within the patient. In at least some embodiments, the sonolucent fluid has an acoustic impedance that is within 5 percent of an acoustic impedance of patient tissue or fluid at or near a target imaging site within the patient. In at least some embodiments, the sonolucent fluid also provides lubrication for the interface between the rotating portions of the imaging core 404 and the sheath 428.

It may be detrimental to the patient for the sonolucent fluid to leak into patient tissues or fluids (e.g., following a breach of the imaging core 404). Thus, it may be an advantage to avoid using excessive amounts of sonolucent fluid. In at least some embodiments, the volume of the sonolucent fluid is no greater than 300 nano-liters. In at least some embodiments, the volume of the sonolucent fluid is no greater than 250 nano-liters. In at least some embodiments, the volume of the sonolucent fluid is no greater than 200 nano-liters.

A magnetic field generated by the windings 422 causes the magnet 420 to rotate along a longitudinal axis of the magnet 420. In at least some embodiments, the longitudinal axis of the magnet 420 is parallel to the longitudinal axis of the imaging core 404. In at least some embodiments, an applied current creates the magnetic field in the windings 422. In some embodiments, the rotation of the magnet 420 causes a corresponding rotation of the one or more transducers 418. In other embodiments, the one or more transducers 418 are fixed and the rotation of the magnet 420 causes a corresponding rotation of a tilted mirror configured and arranged to redirect energy beams (e.g., acoustic signals and echo signals) to and from the one or more fixed transducers 418.

In FIG. 4, the imaging core 404 includes one or more fixed transducers 418 and a signal redirection unit 434 coupled to the magnet 420. In at least some embodiments, the signal redirection unit 434 is coupled to the magnet 420 such that rotation of the magnet 420 causes a corresponding rotation of the signal redirection unit 434. The signal redirection unit 434 includes a tilted mirror 436. In at least some embodiments, the signal redirection unit 434 includes a mount 438 configured and arranged to couple the mirror 436 to the magnet 420. In at least some embodiments, the signal redirection unit 434 includes one or more sonolucent materials 440 disposed over a reflective surface 442 of the mirror 436.

In at least some embodiments, the one or more sonolucent materials 440 have an acoustic impedance that is within 20 percent of an acoustic impedance of patient tissue or fluid at or near a target imaging site within the patient. In at least some embodiments, the one or more sonolucent materials 440 have an acoustic impedance that is within 15 percent of an acoustic impedance of patient tissue or fluid at or near a target imaging site within the patient. In at least some embodiments, the one or more sonolucent materials 440 have an acoustic impedance that is within 10 percent of an acoustic impedance of patient tissue or fluid at or near a target imaging site within the patient. In at least some embodiments, the one or more sonolucent materials 440 have an acoustic impedance that is within 5 percent of an acoustic impedance of patient tissue or fluid at or near a target imaging site within the patient.

In at least some embodiments, the one or more sonolucent materials 440 are formed by a molding process. In at least some embodiments, the one or more sonolucent materials 440, the mirror 436, and the mount 438 are configured and arranged such that the signal redirection unit 434 has an even weight distribution around an axis of rotation of the signal redirection unit 434. In at least some embodiments, the axis of rotation of the signal redirection unit 434 is parallel to the longitudinal axis of the magnet 420. In at least some embodiments, the signal redirection unit 434 is substantially cylindrically shaped.

Any impedance-matching material suitable for insertion into a patient may be used to form one or more of the sheath 428, the sonolucent fluid disposed in the imaging core 404, or the one or more sonolucent materials 440 of the signal redirection unit 434. Examples of suitable impedance-matching materials may include one or more rubbers (e.g., polyurethanes, room temperature vulcanization rubber, or the like or combinations thereof), one or more plastics (e.g., polyethylene, ethyl vinyl acetate, or the like or combinations thereof), one or more liquids (e.g., furfuryl alcohol, butylene glycol, polyethylene glycol, ethylnapthalene, castor oil, linseed oil, paraffin oil, silicon oil, water, salt water, sea water, or the like or combinations thereof), or the like or combinations thereof.

In at least some embodiments, the tilted mirror 436 is angled to redirect acoustic signals emitted from the one or more fixed transducers 418 to a direction that is roughly perpendicular to the longitudinal axis of the magnet 420. In at least some embodiments, the tilted mirror 436 is angled to redirect acoustic signals emitted from the one or more fixed transducers 418 to a direction that is not perpendicular to the longitudinal axis of the magnet 420. In at least some embodiments, the tilted mirror 436 is angled to redirect acoustic signals emitted from the one or more fixed transducers 418 to one or more of a plurality of angles that are within a 120 degree range with respect to a transverse axis of the magnet 420. In at least some embodiments, the tilted mirror 436 is angled to redirect acoustic signals emitted from the one or more fixed transducers 418 to one or more of a plurality of angles that are within a 90 degree range with respect to the transverse axis of the magnet 420.

In at least some embodiments, the tilted mirror 436 has a concave shape that is configured and arranged to focus the ultrasound beam emitted from transducer 418 to a non-diverging beam within the surrounding patient tissues. In at least some embodiments, the mount 438 is formed from one or more materials that attenuate ultrasound energy that may transmit through tilted mirror 436, thus reducing, or even preventing, reflections from distal parts, such as the magnet 420. In at least some embodiments, one or more materials may be disposed between the tilted mirror 436 and the mount 438 that attenuate ultrasound energy that may transmit through tilted mirror 436, thus reducing, or even preventing, reflections from distal parts, such as the magnet 420.

In at least some embodiments, the one or more transducers 418 are disposed at a proximal end of the imaging core 404 and emit acoustic signals distally along the longitudinal axis of the imaging core 404, towards the reflective surface 442 of the tilted mirror 436. It may be an advantage to dispose the one or more transducers 418 at a proximal end of the imaging core 404 so that the one or more transducer conductors 424 do not extend along rotating portions of the imaging core 404 (e.g., the magnet 420, the signal redirection unit 434, or the like).

In at least some embodiments, the magnet 420 is a permanent magnet. The magnet 420 may be formed from many different magnetic materials suitable for implantation including, for example, neodymium-iron-boron, or the like. One example of a suitable neodymium-iron-boron magnet is available through Hitachi Metals America Ltd, San Jose, Calif.

In at least some embodiments, the magnet 420 is cylindrical. In at least some embodiments, the magnet 420 has a magnetization M of no less than 1.4 T. In at least some embodiments, the magnet 420 has a magnetization M of no less than 1.5 T. In at least some embodiments, the magnet 420 has a magnetization M of no less than 1.6 T. In at least some embodiments, the magnet 420 has a magnetization vector that is perpendicular to the longitudinal axis of the magnet 420.

In at least some embodiments, the one or more transducers 418 are ring-shaped. In at least some embodiments, the one or more transducers 418 are C-shaped. In at least some embodiments, the one or more transducers 418 are fixedly coupled to an inner surface of the imaging core 404.

When, as shown in FIG. 4, the one or more transducers 418 are ring-shaped and the guidewire 408 extends through the one or more transducers 418, the guidewire 408 may obstruct some of the acoustic signals from reaching patient tissue, and may also obstruct some of the echo signals from patient tissues from reaching the one or more transducers 418 (i.e., a blind spot may be formed). FIG. 5A is a schematic transverse cross-sectional view of one embodiment of the guidewire 408 extending through the one or more transducers 418. A blind spot 502 is formed over a portion of the one or more transducers 418 because the guidewire 408 obstructs the path of signals transmitting between the one or more transducers 418 and patient tissue. The positioning of the blind spot 502 rotates with the rotation of the magnet (420 in FIG. 4). Arrow 504 shows the direction of the reflected acoustic signals when emitted from the one or more transducers 418 and reflected from the mirror (436 in FIG. 4). As shown in FIG. 5A, when the mirror (436 in FIG. 4) is tilted such that acoustic signals emitted from the one or more transducers 418 are reflected upward, the blind spot 502 is positioned beneath the guidewire 408.

Similarly, as shown in FIG. 5B, when the magnet (420 in FIG. 4) is rotated clockwise ninety degrees, the mirror (436 in FIG. 4) is also rotated clockwise ninety degrees. Accordingly, arrow 506 shows the direction of reflected acoustic signals being to the right. As a result, the blind spot 502 is rotated clockwise ninety degrees to the left of the guidewire 408.

In at least some embodiments, the imaging core 404 has a diameter that is no greater than 1.3 millimeters. In at least some embodiments, the imaging core 404 has a diameter that is no greater than 1.2 millimeters. In at least some embodiments, the imaging core 404 has a diameter that is no greater than 1.1 millimeters. In at least some embodiments, the imaging core 404 has a diameter that is no greater than one millimeter. In at least some embodiments, the imaging core 404 has a diameter that is no greater than 0.9 millimeters. In at least some embodiments, the imaging core 404 has a diameter that is no greater than 0.8 millimeters.

In at least some embodiments, the guidewire 408 has a diameter that is no greater than 0.5 millimeters. In at least some embodiments, the guidewire 408 has a diameter that is no greater than 0.4 millimeters. In at least some embodiments, the guidewire 408 has a diameter that is no greater than 0.3 millimeters. In at least some embodiments, the guidewire 408 has a diameter that is no greater than 0.2 millimeters.

In at least some embodiments, the one or more transducers 418 have an outer diameter no greater than 1.2 millimeters. In at least some embodiments, the one or more transducers 418 have a diameter no greater than 1.1 millimeters. In at least some embodiments, the one or more transducers 418 have a diameter no greater than one millimeter. In at least some embodiments, the one or more transducers 418 have a diameter no greater than 0.9 millimeters. In at least some embodiments, the one or more transducers 418 have a diameter no greater than 0.8 millimeters.

In at least some embodiments, the one or more transducers 418 have an inner diameter no greater than 0.5 millimeters. In at least some embodiments the one or more transducers 418 have an inner diameter no greater than 0.4 millimeters. In at least some embodiments, the one or more transducers 418 have an inner diameter no greater than 0.3 millimeters. In at least some embodiments, the one or more transducers 418 have an inner diameter no greater than 0.2 millimeters.

In preferred embodiments, the windings 422 are formed from rigid or semi-rigid materials using multiple-phase winding geometries. It will be understood that there are many different multiple-phase winding geometries and current configurations that may be employed to form a rotating magnetic field. For example, the windings 422 may include, for example, a two-phase winding, a three-phase winding, a four-phase winding, a five-phase winding, or more multiple-phase winding geometries. It will be understood that a motor may include many other multiple-phase winding geometries. In a two-phase winding geometry, for example, the currents in the two windings are out of phase by 90°. For a three-phase winding, there are three lines of sinusoidal current that are out of phase by zero, 120°, and 240°, with the three current lines also spaced by 120°, resulting in a uniformly rotating magnetic field that can drive a cylindrical rotor magnet magnetized perpendicular to the current lines.

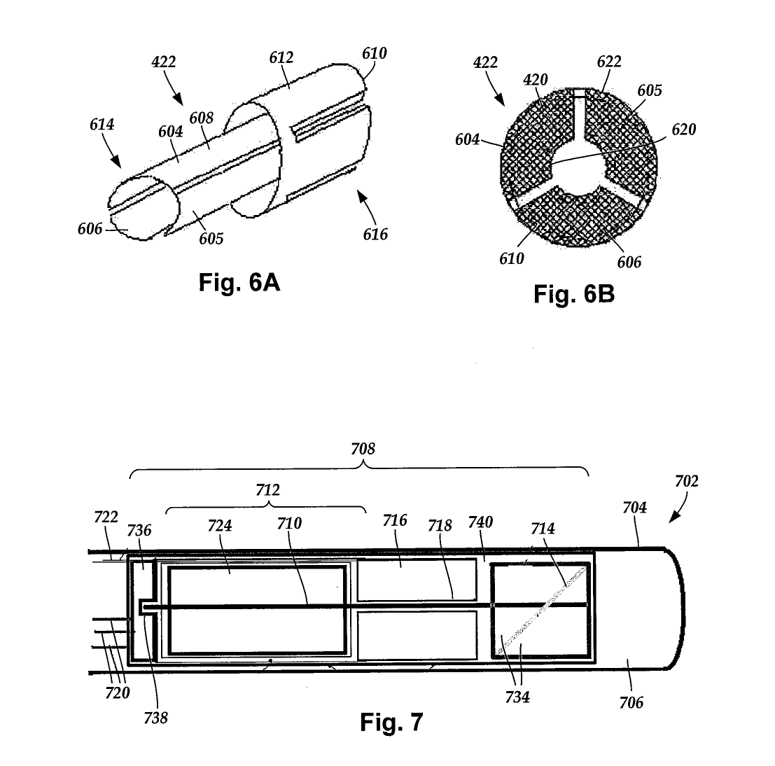

In at least some embodiments, the windings 422 utilize a three-phase winding geometry. FIG. 6A is a schematic perspective view of one embodiment of the windings 422 configured using a three-phase winding geometry for forming a rotating magnetic field around the magnet (420 in FIG. 4). The windings 422 are disposed on three arms 604-606 to form an inner section 608, a radial section 610, and an outer section 612. The windings 422 also include a proximal end 614 and a distal end 616. In at least some embodiments, the windings 422 are configured and arranged to be disposed around both an inner surface and an outer surface of the magnet (420 in FIG. 4), the inner surface at an inner diameter 620 and the outer surface at an outer diameter 622 of the magnet (420 in FIG. 4).

In at least some embodiments, the inner section 608 of the windings 422 extend along a length of the guidewire lumen (410 in FIG. 4) along the inner surface at the inner diameter 620 of the magnet (420 in FIG. 4) to a distal end of the magnet (420 in FIG. 4). In at least some embodiments, at least a portion of the inner section 608 of the windings 422 is integrated into the guidewire lumen 410. The radial section 610 extends radially outward from the inner section 608. In at least some embodiments, the radial section 610 is disposed in proximity to the distal end 616 of the windings 422. In at least some embodiments, the radial section 610 extends along a distal end of the magnet (420 in FIG. 4). In alternate embodiments, the radial section 610 is disposed in proximity to the proximal end 614 of the windings 422 and extends along a proximal end of the magnet (420 in FIG. 4).

The outer section 612 of the windings 422 extends from the radial section 610 of the windings 422. In at least some embodiments, the outer section 612 extends along the outer surface at the outer diameter 622 of the magnet (420 in FIG. 4). It may be an advantage to have both the inner section 608 of the windings 422 extending along the inner surface of the magnet (420 in FIG. 4) and the outer section 612 of the windings 422 extending along the outer surface of the magnet (420 in FIG. 4) because the surface area of the magnet (420 in FIG. 4) over which the windings 422 are disposed is increased. Thus, the current flowing along the windings 422 can be used to provide additional torque for rotating the magnet (420 in FIG. 4) as compared to windings that extend over just one of the diameters of the magnet (420 in FIG. 4).

Although other geometries may also form a rotating magnetic field, the three-phase geometry 602 may have the advantages of allowing for a more compact motor construction than other geometries. In at least some embodiments, the windings are constructed by forming slits in a solid tube, and are equivalent to single-turn coils. By using a single-turn geometry, there are no winding cross-overs, which may result in thinner windings. One property of a three-phase winding geometry 602 is that only two of the three windings disposed on the arms 604-606 need to be driven, while the third winding is a common return that mathematically is equal to the third phase of current. In at least some embodiments, the arms 604-606 may be supported by a substrate to increase mechanical stability. In at least some embodiments, the arms 604-606 are constructed from a solid tube formed from one or more metals or metal alloys (e.g., beryllium-copper, tungsten-copper, or the like), leaving most of the material in tact, and removing only material needed to prevent electrical shorting between the arms 604-606.

In at least some embodiments, the arms 604-606 each have thicknesses of no more than 0.004 inches (0.1 mm). In at least some embodiments, the arms 604-606 each have thicknesses of no more than 0.003 inches (0.08 mm). In at least some embodiments, the arms 604-606 each have thicknesses of no more than 0.002 inches (0.05 mm). In at least some embodiments, the arms 604-606 are formed from a cylindrical material with a plurality of slits are defined along at least a portion of a longitudinal length of each of the arms 604-606. In at least some embodiments, at least one of the slits is back filled with one or more electrically insulating materials, such as an epoxy.

FIG. 6B is a schematic distal end view of one embodiment of the magnet 420 disposed in the winding 422. In FIG. 6B, a plurality of current paths are shown along the radial section 610 of the arms 604-606 from the inner surface of the magnet 420 to the outer surface of the magnet 420, the inner surface being at the inner diameter 620 and the outer surface being at the outer diameter 622.

In alternate embodiments, the imaging core is configured and arranged for insertion into a catheter, but that is not configured and arranged for being guided by a guidewire. In at least some embodiments, the imaging core is disposed in a catheter defining a lumen into which the imaging core can be disposed and at least partially pulled back there through during an imaging procedure, as discussed above with reference to FIG. 3.

In at least some embodiments, the imaging core includes a fixed transducer and a rotating mirror disposed in a lumen of a catheter. FIG. 7 is a schematic longitudinal cross-sectional view of another embodiment of a distal end of a catheter 702. The catheter 702 includes a sheath 704 and a lumen 706. A sealed and preferably watertight imaging core 708 is disposed in the lumen 706 at the distal end of the catheter 702. In at least some embodiments, a sonolucent fluid is disposed in the imaging core to displace air pockets within the imaging core 708.

In at least some embodiments, the sonolucent fluid has an acoustic impedance that is within 20 percent of an acoustic impedance of patient tissue or fluid at the ultrasound imaging frequency, at or near a target imaging site within the patient. In at least some embodiments, the sonolucent fluid has an acoustic impedance that is within 15 percent of an acoustic impedance of patient tissue or fluid at the ultrasound imaging frequency, at or near a target imaging site within the patient. In at least some embodiments, the sonolucent fluid has an acoustic impedance that is within 10 percent of an acoustic impedance of patient tissue or fluid at the ultrasound imaging frequency, at or near a target imaging site within the patient. In at least some embodiments, the sonolucent fluid has an acoustic impedance that is within 5 percent of an acoustic impedance of patient tissue or fluid at the ultrasound imaging frequency, at or near a target imaging site within the patient. In at least some embodiments, the sonolucent fluid also provides lubrication for the interface between the rotating portions of the imaging core 404 and the sheath 428.

The imaging core 708 includes a rotatable driveshaft 710 with a motor 712 and a mirror 714 coupled to the driveshaft 710 and configured and arranged to rotate with the driveshaft 710. In at least some embodiments, the mirror 714 is part of a signal redirection unit 734. The imaging core 708 also includes one or more transducers 716 defining an aperture 718 extending along a longitudinal axis of the one or more transducers 716. In at least some embodiments, the one or more transducers 716 are positioned between the motor 712 and the mirror 714. In at least some embodiments, the one or more transducers 716 are configured and arranged to remain stationary while the driveshaft 710 rotates. In at least some embodiments, the driveshaft 710 extends through the aperture 718 defined in the one or more transducers 716. In at least some embodiments, the aperture 718 is formed from a material, or includes a coating, or both, such as polytetrafluoroethylene coated polyimide tubing, that reduces drag between the rotatable driveshaft 710 and the stationary (relative to the driveshaft 710) aperture 718 of the one or more transducers 716.

One or more motor conductors 720 electrically couple the motor 712 to the control module (104 in FIG. 1). In at least some embodiments, the one or more of the motor conductors 720 may extend along at least a portion of the longitudinal length of the catheter 702 as braided electrical cables, or as shielded, twisted pair cable, or the like. One or more transducer conductors 722 electrically couple the one or more transducers 716 to the control module (104 in FIG. 1). In at least some embodiments, the one or more of the catheter conductors 722 may extend along at least a portion of the longitudinal length of the catheter 702 as shielded electrical cables, such as a coaxial cable, or a twisted pair cable, or the like.

The magnet 724 is coupled to the driveshaft 710 and is configured and arranged to rotate the driveshaft 710 during operation. In at least some embodiments, the magnet 724 is rigidly coupled to the driveshaft 710. In at least some embodiments, the magnet 724 is coupled to the driveshaft 710 by an adhesive.

In at least some embodiments, the imaging core 708 includes a proximal end cap 736. In at least some embodiments, the proximal end cap 736 provides structure to the proximal portion of the imaging core 708. In at least some embodiments, the proximal end cap 736 is rigid enough to withstand lateral forces (i.e., off-axis forces) typically encountered during normal operation within patient vasculature such that the operation of the motor 712 is not interrupted. In at least some embodiments, a proximal end of the driveshaft 710 contacts the proximal end cap 736. In at least some embodiments, the proximal end cap 736 defines a drag-reducing element 738 for reducing drag caused by the rotating driveshaft 710 contacting the proximal end cap 736. The drag-reducing element 738 can be any suitable device for reducing drag including, for example, one or more bushings, one or more bearings, or the like or combinations thereof. In at least some embodiments, the drag-reducing element 738 facilitates uniformity of rotation of the driveshaft 710.

In at least some embodiments, the imaging core 708 is sealed by an inner sheath 740. In at least some embodiments, the inner sheath 740 is rigid. In at least some embodiments, the inner sheath 740 is rigid enough to withstand lateral forces (i.e., off-axis forces) typically encountered during normal operation within patient vasculature such that the mirror 714 does not contact the inner sheath 740. In at least some embodiments, the inner sheath 740 is filled with a sonolucent fluid. In at least some embodiments, the one or more motor conductors 720 and the one or more catheter conductors 722 extend through the inner sheath 740.

In at least some embodiments, the sonolucent fluid within the sealed and watertight imaging core 708 has an acoustic impedance that is within 20 percent of an acoustic impedance of patient tissue or fluid at the ultrasound imaging frequency, at or near a target imaging site within the patient. In at least some embodiments, the sonolucent fluid within the sealed and watertight imaging core 708 has an acoustic impedance that is within 15 percent of an acoustic impedance of patient tissue or fluid at the ultrasound imaging frequency, at or near a target imaging site within the patient. In at least some embodiments, the sonolucent fluid within the sealed and watertight imaging core 708 has an acoustic impedance that is within 10 percent of an acoustic impedance of patient tissue or fluid at the ultrasound imaging frequency, at or near a target imaging site within the patient. In at least some embodiments, the sonolucent fluid within the sealed and watertight imaging core 708 has an impedance that is within 5 percent of an acoustic impedance of patient tissue or fluid at the ultrasound imaging frequency, at or near a target imaging site within the patient.

In at least some embodiments, the wall thickness of the sheath 720 is less than the wavelength of an ultrasound beam transmitting in patient fluid or tissue at the imaging frequency of the one or more transducers 716. In some embodiments, the wall thickness of the sheath 720 is no more than, or less than one-half of the wavelength of an ultrasound beam transmitting in patient fluid or tissue at the imaging frequency of the one or more transducers 716. In some embodiments, the wall thickness of the sheath 720 is no more than, or less than one-quarter of the wavelength of an ultrasound beam transmitting in patient fluid or tissue at the imaging frequency of the one or more transducers 716.

Without wishing to be held to any particular values, in one example ultrasound signals transmitting from the one or more transducers 418 have a frequency of 40 MHz, and the speed of sound in surrounding tissue is 1,500 m/sec, so the ultrasound wavelength is about 0.04 mm. In at least some embodiments, when the ultrasound wavelength is about 0.04 mm, the sheath 428 is constructed from polyimide having a wall thickness of no more than 0.04 mm. In at least some embodiments, when the ultrasound wavelength is about 0.04 mm, the sheath 428 is constructed from polyimide having a wall thickness of no more than 0.02 mm. In at least some embodiments, when the ultrasound wavelength is about 0.04 mm, the sheath 428 is constructed from polyimide having a wall thickness of no more than 0.01 mm.

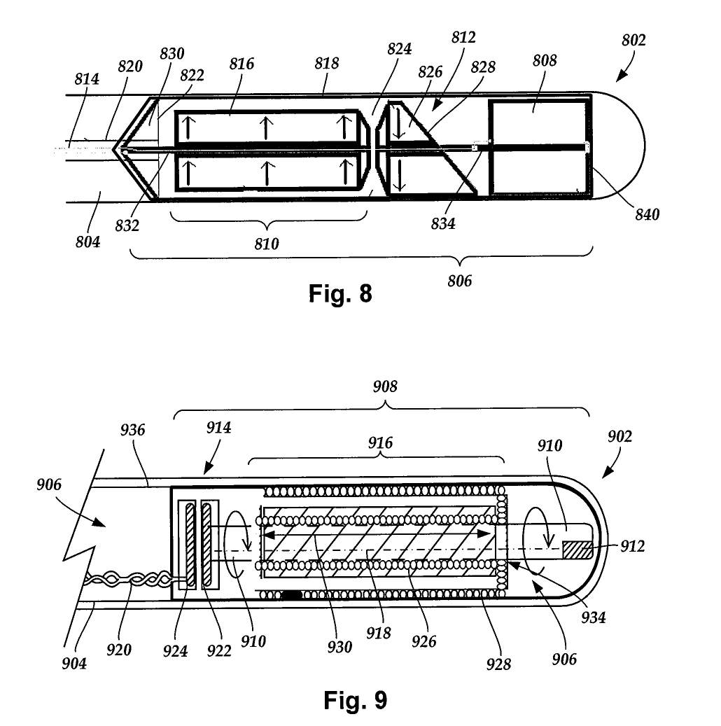

In at least some embodiments, the rotatable mirror is positioned proximal to the one or more fixed transducers. FIG. 8 is a schematic longitudinal cross-sectional view of another embodiment of a distal end of a catheter 802. The catheter 802 defines a lumen 804 within which a sealed and preferably watertight imaging core 806 is disposed. The imaging core 806 is sealed by a sheath 840. The imaging core 806 includes one or more fixed transducers 808, a motor 810, and a rotating mirror 812 proximal to the one or more transducers 808. The one or more transducers 808 electrically couple to the control module (104 in FIG. 1) via one or more transducer conductors 814. In at least some embodiments, the one or more transducer conductors 814 extend through the sheath 840.

The motor 810 includes a rotatable magnet 816 and windings 818. The windings 818 are provided with power to generate a magnetic field from the control module (104 in FIG. 1) via one or more stator conductors 820. In at least some embodiments, the motor 810 is disposed in a housing 822 with a distal end cap 824. In at least some embodiments, the one or more stator conductors 820 extend through the sheath 840.

The mirror 812 includes a magnet 826 and a tilted reflective surface 828. In at least some embodiments, the mirror 812 is configured and arranged to rotate with the magnet 816. In at least some embodiments, the mirror 812 is not mechanically coupled to the end cap 824. The magnet 816 is magnetically coupled to the mirror 812 through the end cap 824.

The end cap 824 can be formed from a rigid or semi-rigid material (e.g., one or more metals, alloys, plastics, composites, or the like). In at least some embodiments, the end cap 824 is coated with a slick material (e.g., polytetrafluoroethylene, or the like) to reduce friction between the end cap 824 and the rotating magnet 816 and mirror 812. In at least some embodiments, at least one of the magnet 816 or the mirror 812 has a tapered end contacting the end cap 824 to reduce friction during rotation.

In at least some embodiments, the imaging core 806 includes a support hub 830 disposed at a distal end of the imaging core 806. In at least some embodiments, the windings 818 are supported on one end by the support hub 830 and on the opposite end by the end cap 824. In at least some embodiments, the motor 810 includes a motor shaft 832 providing a longitudinal axis about which the magnet 816 rotates. In at least some embodiments, the motor shaft 832 is coupled on one end by the support hub 830 and on the opposite end by the end cap 824. In at least some embodiments, the one or more transducers 808 are coupled to a transducer shaft 834 extending distally from the end cap 824. In at least some embodiments, the mirror 812 defines an aperture through which the transducer shaft 834 extends. In at least some embodiments, the one or more transducer conductors 814 are at least partially disposed in the transducer shaft 834. In at least some embodiments, the one or more transducer conductors 814 are at least partially disposed in the motor shaft 832. In alternate embodiments, the one or more transducer conductors 814 extend around an outer surface of one or more of the motor 810 or the mirror 812.

In at least some embodiments, the imaging core includes one or more rotatable transducers. FIG. 9 is a schematic longitudinal cross-sectional view of one embodiment of a distal end of a catheter 902. The catheter 902 includes a sheath 904 and a lumen 906. A sealed and preferably watertight imaging core 908 is disposed in the sheath 904. The sheath 904 is disposed in the lumen 906 at the distal end of the catheter 902. The imaging core 908 includes a rotatable driveshaft 910 with one or more transducers 912 coupled to a distal end of the driveshaft 910 and a transformer 914 coupled to a proximal end of the driveshaft 910. The imaging core 908 also includes a motor 916 coupled to the driveshaft 910. One or more imaging core conductors 918 electrically couple the one or more transducers 912 to the transformer 914. In at least some embodiments, the one or more imaging core conductors 918 extend within the driveshaft 910. One or more catheter conductors 920 electrically couple the transformer 914 to the control module (104 in FIG. 1). In at least some embodiments, the one or more of the catheter conductors 920 may extend along at least a portion of a length of the catheter 902 as shielded electrical cables, such as a coaxial cable, or a twisted pair cable, or the like. In at least some embodiments, the one or more catheter conductors 920 extend through the sheath 904.

The transformer 914 is disposed on the imaging core 908. In at least some embodiments, the transformer 914 includes a rotating component 922 coupled to the driveshaft 910 and a stationary component 924 disposed spaced apart from the rotating component 914. In some embodiments, the stationary part 924 is proximal to, and immediately adjacent to, the rotating component 922. The rotating component 922 is electrically coupled to the one or more transducers 912 via the one or more imaging core conductors 918 disposed in the imaging core 908. The stationary component 916 is electrically coupled to the control module (104 in FIG. 1) via one or more conductors 920 disposed in the lumen 906. Current is inductively passed between the rotating component 922 and the stationary component 924 (e.g., a rotor and a stator, or a rotating pancake coil and a stationary pancake coil, or the like).

In at least some embodiments, the transformer 914 is positioned at a proximal end of the imaging core 908. In at least some embodiments, the components 922 and 924 of the transformer 914 are disposed in a ferrite form. In at least some embodiments, the components 922 and 924 are smaller in size than components conventionally positioned at the proximal end of the catheter.

The motor 916 includes a magnet 926 and windings 928. In at least some embodiments, the magnet 926 is a permanent magnet with a longitudinal axis, indicated by a two-headed arrow 930, which is coaxial with the longitudinal axis of the imaging core 908 and the driveshaft 910.

In at least some embodiments, the magnet 926 is coupled to the driveshaft 910 and is configured and arranged to rotate the driveshaft 910 during operation. In at least some embodiments, the magnet 926 defines an aperture 934 along the longitudinal axis 930 of the magnet 926. In at least some embodiments, the driveshaft 910 and the one or more imaging core conductors 918 extend through the aperture 934. In at least some other embodiments, the drive shaft 910 is discontinuous and, for example, couples to the magnet 926 at opposing ends of the magnet 926. In which case, the one or more imaging core conductors 918 still extend through the aperture 934. In at least some embodiments, the magnet 926 is coupled to the driveshaft 910 by an adhesive. Alternatively, in some embodiments the driveshaft 910 and the magnet 926 can be machined from a single block of magnetic material with the aperture 934 drilled down a length of the driveshaft 910 for receiving the imaging core conductors 918. The windings 928 are provided with power from the control module (104 in FIG. 1) via one or more motor conductors 936. In at least some embodiments, the one or more motor conductors 936 extend through the sheath 904.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A catheter assembly for an intravascular ultrasound system, the catheter assembly comprising:
a catheter having a length, a distal end, and a proximal end, wherein the distal end is configured and arranged for insertion into patient vasculature;
a sealed imaging core disposed in the distal end of the catheter, the sealed imaging core configured and arranged to provide a watertight environment within the sealed imaging core, the sealed imaging core having a proximal end, a distal end, and a length that is substantially less than the length of the catheter, the sealed imaging core comprising
a motor comprising a magnet and at least two magnetic field windings, wherein the magnet is configured and arranged to rotate upon generation of a magnetic field by the at least two magnetic field windings,
at least one fixed transducer configured and arranged for transforming applied electrical signals to acoustic signals, transmitting the acoustic signals, receiving corresponding echo signals, and transforming the received echo signals to electrical signals,
a tilted mirror coupled to the magnet such that rotation of the magnet causes a corresponding rotation of the tilted mirror, the tilted mirror configured and arranged to redirect acoustic signals transmitted from the at least one fixed transducers to patient tissue, and
at least one sonolucent fluid disposed in the sealed imaging core, the at least one sonolucent fluid filling open space within the sealed imaging core;
at least one transducer conductor electrically coupled to the at least one fixed transducer within the sealed imaging core, wherein the at least one transducer conductor extends from the at least one fixed transducer to a location that is external to the sealed imaging core; and
at least one stator conductor electrically coupled to the magnetic field windings within the sealed imaging core, wherein the at least one stator conductor extends from the magnetic field windings to a location that is external to the sealed imaging core.

2. The catheter assembly of claim 1, wherein the distal end is configured and arranged for insertion into patient vasculature via a guidewire.

3. The catheter assembly of claim 2, wherein the magnet has an inner surface at an inner diameter and an outer surface at an outer diameter, the inner diameter defined by an aperture along a longitudinal axis of the at least one fixed transducer, the magnet aperture configured and arranged to allow passage of the guidewire, the at least two magnetic field windings disposed around at least a portion of both the inner surface and the outer surface of the magnet.

4. The catheter assembly of claim 2, wherein the at least one transducer has an aperture defined along a longitudinal axis of the at least one fixed transducer, the at least one fixed transducer aperture configured and arranged to allow passage of the guidewire.

5. The catheter assembly of claim 1, wherein the at least one sonolucent fluid disposed in the imaging core is in a quantity of no more than 300 nano-liters.

6. The catheter assembly of claim 1, wherein at least one sonolucent material is disposed in the sealed imaging core between the tilted mirror and the at least one fixed transducer.

7. The catheter assembly of claim 6, wherein the sealed imaging core consists essentially of the motor, the at least one fixed transducer, the tilted mirror, the at least one sonolucent material disposed between the tilted mirror and the at least one transducer, and the at least one sonolucent fluid filling open space within the sealed imaging core.

8. An intravascular ultrasound imaging system comprising:
the catheter assembly of claim 1; and
a control module coupled to the imaging core, the control module comprising
a pulse generator configured and arranged for providing electric signals to the at least one fixed transducer, the pulse generator electrically coupled to the at least one fixed transducer via the at least one transducer conductor, and
a processor configured and arranged for processing received electrical signals from the at least one fixed transducer to form at least one image, the processor electrically coupled to the at least one fixed transducer via the at least one transducer conductor.

9. A catheter assembly for an intravascular ultrasound system, the catheter assembly comprising:
a catheter having a length, a distal end, and a proximal end, wherein the distal end is configured and arranged for insertion into patient vasculature via a guidewire;
an imaging core disposed in the distal end of the catheter, the imaging core configured and arranged to provide a watertight environment within the imaging core, the imaging core having a proximal end, a distal end, and a length that is substantially less than the length of the catheter, the imaging core comprising
a motor comprising a magnet and at least two magnetic field windings, wherein the magnet is configured and arranged to rotate upon generation of a magnetic field by the at least two magnetic field windings, the magnet having an inner surface at an inner diameter and an outer surface at an outer diameter, the inner diameter defined by an aperture along a longitudinal axis of the at least one transducer, the magnet aperture configured and arranged to allow passage of the guidewire, the at least two magnetic field windings disposed around at least a portion of both the inner surface and the outer surface of the magnet,
at least one fixed transducer having an aperture defined along a longitudinal axis of the at least one transducer, the at least one fixed transducer aperture configured and arranged to allow passage of the guidewire, the at least one fixed transducer configured and arranged for transforming applied electrical signals to acoustic signals, transmitting the acoustic signals, receiving corresponding echo signals, and transforming the received echo signals to electrical signals, and
a signal redirection unit coupled to the magnet such that rotation of the magnet causes a corresponding rotation of at least a portion of the signal redirection unit, the signal redirection unit comprising a tilted mirror configured and arranged to redirect acoustic signals transmitted from the at least one fixed transducers to patient tissue;
at least one transducer conductor electrically coupled to the at least one transducer and in electrical communication with the proximal end of the catheter; and
at least one stator conductor electrically coupled to the magnetic field windings and in electrical communication with the proximal end of the catheter.

10. The catheter assembly of claim 9, wherein the at least one fixed transducer is positioned at the proximal end of the imaging core.

11. The catheter assembly of claim 9, further comprising a sonolucent sheath disposed radially around the imaging core.

12. The catheter assembly of claim 9, further comprising at least one sonolucent fluid disposed in the imaging core.

13. The catheter assembly of claim 12, wherein the at least one sonolucent fluid disposed in the imaging core is in a quantity of no more than 300 nano-liters.

14. The catheter assembly of claim 9, wherein the signal redirection unit comprises at least one sonolucent material disposed between the at least one fixed transducer and the tilted mirror.

15. The catheter assembly of claim 9, wherein the catheter defines
a first guidewire port defined in the catheter, the first guidewire port configured and arranged to receive the guidewire;
a second guidewire port defined in the catheter, the second guidewire port configured and arranged to receive the guidewire; and
a guidewire lumen defined along at least a portion of the catheter, the guidewire lumen extending from the first guidewire port to the second guidewire port and arranged to receive the guidewire.

16. The catheter assembly of claim 9, wherein a portion of the at least two magnetic field windings are integrated into the guidewire lumen.

17. An intravascular ultrasound imaging system comprising:
the catheter assembly of claim 9; and
a control module coupled to the imaging core, the control module comprising
a pulse generator configured and arranged for providing electric signals to the at least one fixed transducer, the pulse generator electrically coupled to the at least one fixed transducer via the at least one transducer conductor, and
a processor configured and arranged for processing received electrical signals from the at least one fixed transducer to form at least one image, the processor electrically coupled to the at least one fixed transducer via the at least one transducer conductor.

18. A method for imaging a patient using an intravascular ultrasound imaging system, the method comprising:
inserting a catheter into patient vasculature, the catheter comprising a sealed, watertight imaging core coupled to the guidewire, the imaging core electrically coupled to a control module by at least one transducer conductor, the imaging core having at least one fixed transducer and a magnet that rotates by application of a magnetic field generated from at least two magnetic field windings, wherein the magnet has an inner surface at an inner diameter and an outer surface at an outer diameter, wherein the at least two magnetic field windings are disposed around at least a portion of both the inner surface and the outer surface of the magnet, and wherein the transducer emits acoustic signals directed at a tilted mirror configured and arranged to rotate with the magnet and redirect the acoustic signals to patient tissue;
transmitting at least one electrical signal from the control module to the at least one transducer;
generating a magnetic field to cause the magnet to rotate;
transmitting at least one acoustic signal from the at least one transducer to the tilted mirror;
redirecting at least one echo signal received from a tissue-boundary between adjacent imaged patient tissue to the at least one transducer by the tilted mirror; and
transmitting at least one transformed echo signal from the at least one transducer to the control module for processing.

19. The method of claim 18, wherein inserting the catheter into patient vasculature comprises inserting the catheter into patient vasculature via a guidewire.

20. The method of claim 18, wherein inserting the catheter into patient vasculature comprises inserting the catheter into patient vasculature, wherein the catheter defines a lumen configured and arranged to receive the imaging core.
